(19) United States
(12) Patent Application Publication
(10) Pub. No.: US 2016/0145560 A1
(43) Pub. Date: May 26, 2016

(54) DEVICE FOR MICROBIAL CULTIVATION WITH OPTIONS FOR FIELD STERILIZATION AND GAS GENERATION

(71) Applicant: Eino Elias HAKALEHTO, Kuopio (FI)

(72) Inventor: Eino Elias Hakalehto, Kuopio (FI)

(21) Appl. No.: 14/902,529

(22) PCT Filed: Jul. 1, 2014
(86) PCT No.: PCT/FI2014/000014

§ 371 Date: Dec. 31, 2015

(30) Foreign Application Priority Data
Jul. 1, 2013 (FI) U20134158

(51) Int. Cl. C12M 1/12; C12M 1/00

(52) U.S. Cl. CPC C12M 37/00; C12M 23/28; C12M 41/14; C12M 37/02

(57) ABSTRACT

A microbiological cultivation method, and a device for its use is equipped with field-capable system for producing optimal culture conditions for aerobic, microaerobic and anaerobic microbes. They are further equipped with possibilities for independent clean up and sterilization, as well as for gas supply, generation and regulation. This device and method can be used for clinical, environmental and industrial microbial monitoring tasks.

### FIELD OF THE INVENTION

The invention relates to microbial cultivation and especially to portable devices for microbial cultivation, both in the laboratories and in the field.

### BACKGROUND OF THE INVENTION

A microbial detection process typically comprises the following stages: sampling, transportation, pre-cultivation, cultivation, and observation. With the aid of the PMEU-case (Portable Microbial Enrichment Unit) the transportation stage can be left out, which allows for microbial cultivation and observation on the sampling location. This can prevent contamination of the sample during transportation.

Currently the PMEU-case is sterilised by disinfecting with the help of UV-light between the observational periods. The PMEU-case enables microbial cultivation and diagnosis even in difficult environmental conditions, such as crisis areas. In such circumstances, however, maintaining the sterility of the PMEU-case is challenging.

Also, when specific gas atmospheres, and gas mixtures and flow compositions, are used in the PMEU, this requires a system for gas supply. Since pressurized gas bottles are not always available or possible to be used, other techniques are needed for their replacement.

### BRIEF DESCRIPTION OF THE INVENTION

The objective of the invention is thus to develop the device in such a way that the above-mentioned problems are solved. This is achieved with devices and arrangements, which are characterised by what is mentioned in the independent patent claims. The preferred embodiments are the target of the independent claims.

The invention is based on the fact that offered is a microbial cultivation device that comprises the bottom part of the microbial cultivation device, which comprises a syringe holder, which is configured to receiving one or several syringes; the top part of the microbial cultivation device, each top part comprises:
one or more LED sensors and/or similar optical sensors for diagnosis of a sample in corresponding one or more sampling syringes.
thermoregulation unit for controlling the ambient temperature in the sampling syringes.
aeration or gas supply unit for aerating the sampling syringes, or for leading a specific gas mixture into them, and
I/O unit to provide a computer connection between the microbial cultivation device and a computer; wherein the top part is connected to the bottom part so that the top part is removable from the bottom part allowing for the disinfection of the top part and the disposal of the bottom part.

Additionally, a bottom part for a microbial cultivation device is offered for the microbial cultivation device.

Furthermore, a top part for a microbial cultivation device is offered for the microbial cultivation device.

Furthermore a disinfection device comprising tools for receiving the top part of the microbial cultivation device; tools for directing disinfectant and/or hot steam on the received top part of the microbial cultivation device for the disinfection of the microbial cultivation device and/or tools for directing UV-light at the received top part of the microbial cultivation device for disinfection of the top part of the microbial cultivation device.

The advantage of the arrangement according to the invention is that with the help of the invention the sterility of the portable microbial cultivation and observation device can be ensured even under difficult environmental conditions.

### BRIEF DESCRIPTION OF THE DRAWINGS AND THE PHOTOGRAPHS

Referring to the attached drawings, the invention will now be described in greater detail in connection with the preferred embodiments, of which:

FIG. 1 shows a light version of a PMEU device for microbial cultivation or diagnosis according to an exemplary embodiment of the present invention.

FIG. 2 shows a bath for the disinfection of the top part of the simple version of the PMEU device according to an exemplary embodiment of the present invention.

FIG. 3 shows the gas filter of the PMEU light device according to an exemplary embodiment of the present invention.

FIG. 4 shows the gas filter connector of the PMEU light device according to an exemplary embodiment of the present invention.

FIG. 5 shows the cultivation syringe of the PMEU light device according to an exemplary embodiment of the present invention.

FIGS. 6-9 are photographs illustrating the use of a field-capable gas production and supply system. The explanative texts for these figures:

FIG. 6. 30 pieces of 3,5 I CampyGen gas sachets (for 3.5 liters of gas mixture) were added into the sack (110 I). The sack was be closed air-tightly with tape.

FIG. 7. Air is pumped into the sack through the valve.

FIG. 8. The sack is connected to the PMEU and gas flow adjusted.

FIG. 9. In the sack there was enough gas-air mixture for 24 hours of microearobic cultivation in the ten enrichment syringes of the PMEU Spectrion®.

### DETAILED DESCRIPTION OF THE INVENTION

As according to an exemplary embodiment, a light version of the PMEU device (PMEU light) is offered for microbial cultivation and diagnosis (for example in medical diagnostics and in field monitoring for preventing epidemics). The top part of the PMEU light device can be disinfected with UV-light, disinfectant gas (such as ethylene oxide, formaldehyde, vaporised disinfectant (chlorite, etc.), and/or hot steam in a disinfectant bath device which is configured to holding the top part. The top part of the PMEU light device can thus be removed from the bottom part, sterilised, and reused. The top part contains electronics (‘intelligence’) and is therefore the more expensive part of the device. The bottom part of the device contains sampling syringes and a holder for the syringes. The bottom part with the syringes and syringe holder is the cheaper part of the device. The bottom part of the device is disposable and has to be discarded after use. The bottom part does not have to be disinfected and by using a new, unused bottom part for the next cultivation and diagnosis phase the sterility is assured.

Transportation of the samples increases the stress of the cells and thus affects the authenticity and representativeness of the samples. The light version of the PMEU device is a microbiological field laboratory. It speeds up the determination of the microbes and with its help even weak microbes become more visible. The efficient enrichment of the cells started immediately after the sampling, gives a better picture of the situation. The device is suitable for many kinds of research, such as environmental research (i.e. microbe distribution surveying in effluent from wastewater treatment plants), different supervisory tasks in the food industry, wood processing, and clinical research (for example in hospitals). Hidden hygienic risks that do not necessarily show up during regular monitoring can be found with the aid of PMEU.

The PMEU light device enables a real time (online) monitoring of the microbiological quality of water, wherein the device preforms a qualitative analysis of the water samples so that the results are available in real time on a computer. The device can also be configured to send e-mail or text message alerts if E. coli bacteria and/or coliforms are found in the water samples.

Besides environmental operations, the light PMEU system can be used for analysis in clinical microbiology, where it can be applied for patient sample monitoring, or for studying samples from the healthcare environment. Since this system can be supplied with appropriate gas flow for the investigated organisms, it can be used for studying aerobic, microaerobic or anaerobic species of micro-organisms.

In addition to the clinical and environmental monitoring, the PMEU applications can be used also for food safety analysis, and for various hygiene monitoring tasks in different industries. In all applications, the PMEU units can be remote-controlled, and transfer data wirelessly, if needed. This is an option which can be built in the PMEU devices.

The PMEU light device also enables microbial cultivation and diagnosis in harsh environmental conditions such as in crisis areas (for example earthquake, flood, war, or epidemic zones, or refugee camps).

The PMEU light device allows the growth of microbes and determination of deaths in original samples and in original environmental conditions without artificial isolation of bacteria, and the results are available quickly.

Optical measurements can be carried out in an infrared zone (IR), a visible light zone, and an ultraviolet zone (UV). It is also possible to preform a VOC (Volatile Organic Compounds) measurement on the PMEU exhaust gases. Also other measurement methods, such as electrical conductivity measurements or calorimetric measurements, can be carried out.

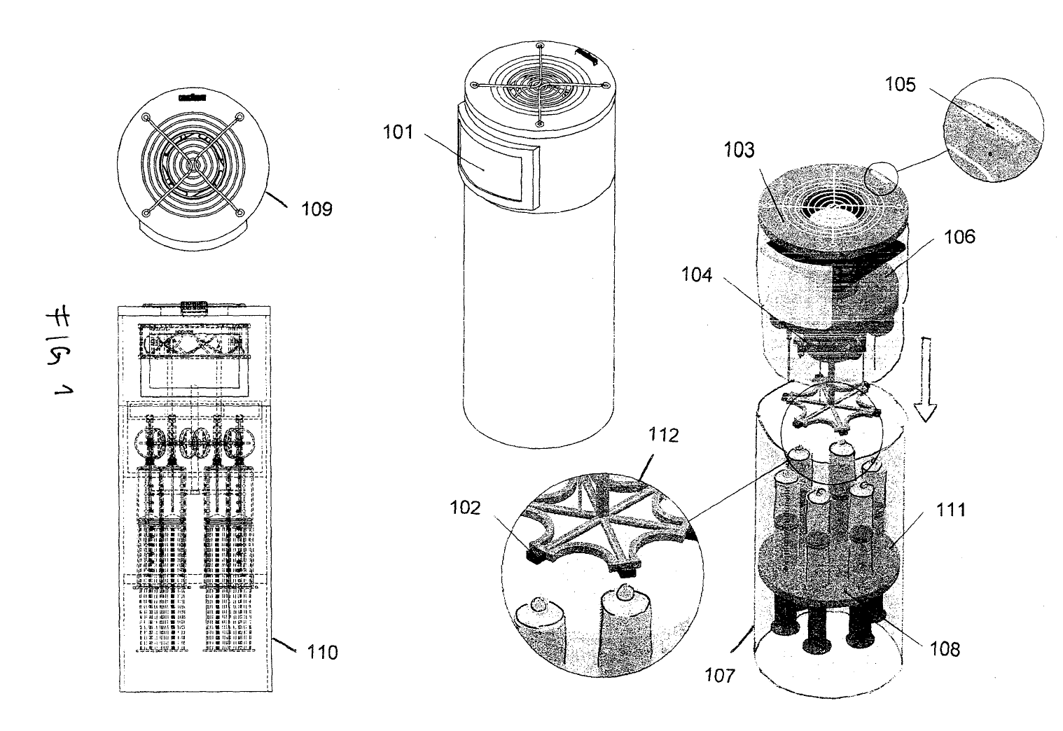

FIG. 1 shows the light version of the PMEU device (PMEU light) for microbial cultivation and diagnosis according to an exemplary embodiment of the present invention. In FIG. 1 the LED sensors 102 or similar optical sensors are shown firmly mounted on the sensor bracket 112 on the top part of the device 106 passing through the centre part of the device. The sensor bracket 112 for the LED sensors and/or similar optical sensors can be customised for different sizes of sampling syringes 108. The syringes 108 are located in the syringe holder 111 on bottom part 107 of the device. The bottom part can also comprise gas filters and corresponding connectors (not shown in FIG. 1). The sensors can analyse the samples in the syringes by analysing for example the microbe growth, density, colour, infrared emission, amount, and/or UV-fluorescence of the samples.

The exemplary embodiment of the device is two-pieced in such a way that a) cultivation syringes (i.e. sampling syringes) 108 along with syringe holders 112 are destroyed due to risk of contamination among other things (in other words, the bottom part 107 of the device, which contains the cultivation syringes 108 with syringe holders 111, is disposable) and b) the more valuable parts of the device, such as the gas exchange unit (i.e. the aeration unit 104), sensors 102, the 112, thermoregulation unit (cooling/heating unit 103), and the control unit (i.e. control tools 101), can be disinfected in a separate disinfection bath (i.e. the top part of the device 106 is reusable).

According to an exemplary embodiment the device is cylindrical in shape. The technical production of a cylindrical shape is affordable. The dimensions of the device may depend on the size (volume) of the samples that are to be processed in the device, which determines the size of the syringes 108 as well as the external dimensions. The device can be manufactured to fit one sample/syringe size or it can be manufactured in a way that different sizes of syringe holders 111 can be inserted in the same device. The top part 106 of the device can at least be partially placed in a protective encasing so that it can be disinfected with hot steam, disinfection gas, and/or UV-light in the disinfection device. This allows for an effective disinfection without disturbing the sensitive electronic parts.

An exemplary embodiment of the microbial cultivation device comprises an input/output I/O unit 105 to provide a telecommunication link between the device and a laptop, PC, and/or mobile phone.

FIG. 1 also shows an exemplary top view 109 seen from above of the microbial cultivation device, and an exemplary cross-sectional side view 110 seen vertically from the side of the device.

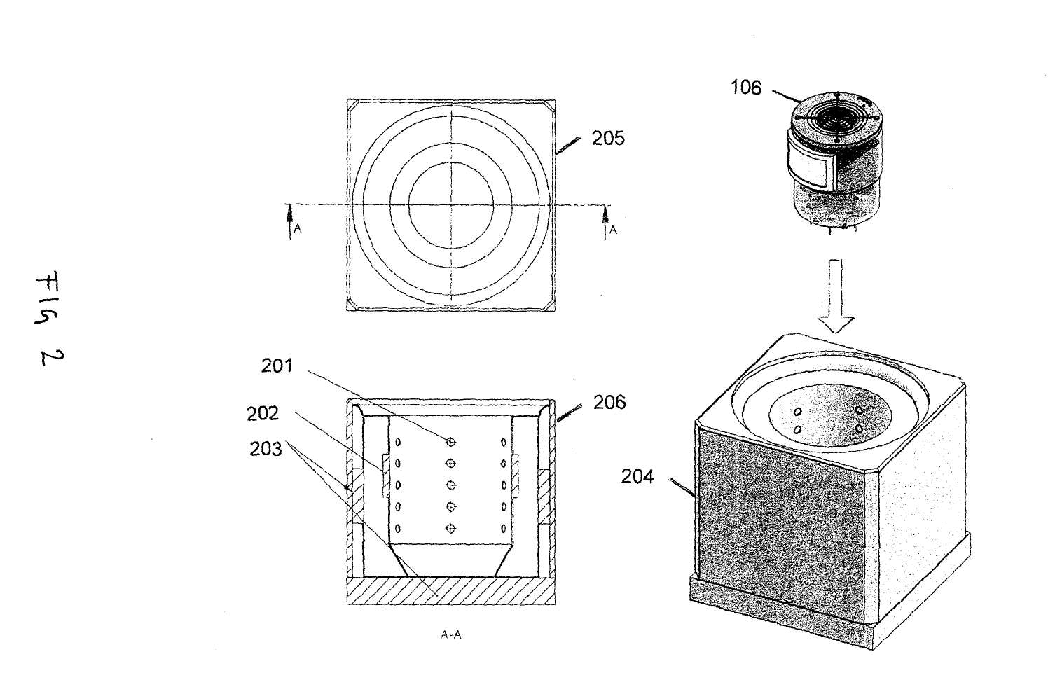

FIG. 2 shows the disinfectant bath for the disinfection of the top part of the PMEU light device according to an exemplary embodiment of the present invention. The disinfection unit 204, 205, 206, i.e. the disinfection device, the so-called disinfection bath, can be based on hot steam, UV-light, disinfection gases, and/or volatile compounds, and/or use of other non-damaging technologies on the top part 106 components. Additionally or alternatively the disinfection device 204, 205, 206 can comprise tools 202 for directing UV-light on the top part 106 of the microbial cultivation device so that the top part 106 can be disinfected with UV-light while the top part is in the disinfection device 104, 205, 206. Additionally or alternatively the disinfection device 204, 205, 206 may comprise a heating element 203 for heating the disinfection device and/or producing hot steam (wherein the steam is produced in a pot and does not come from outside) for the disinfection of the top part 106 of the microbial cultivation device. Pipettes, surgical knives, and/or other medical or laboratory instruments can also be sterilised in the disinfection device 204, 205, 206.

FIG. 2 also shows an exemplary top view 205 of the disinfection device seen from above and an exemplary cross-sectional view 206 along line A-A.

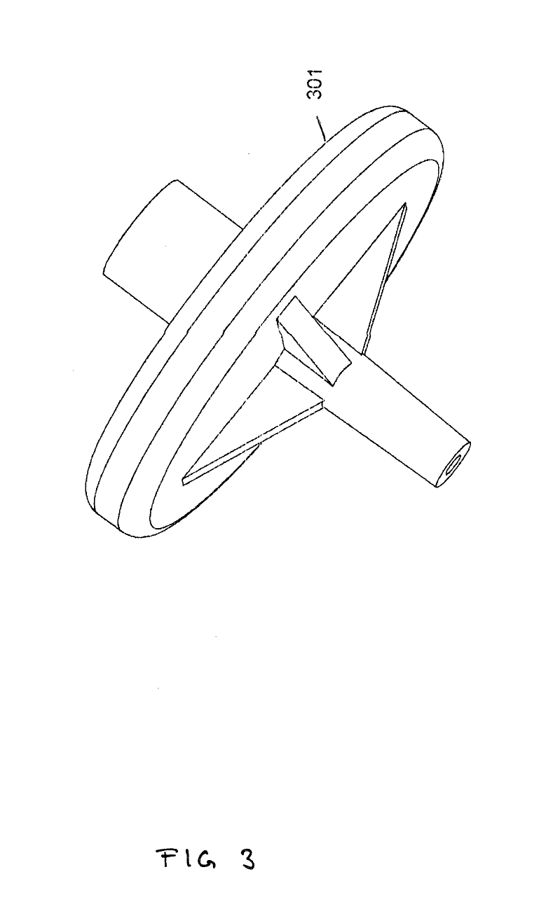

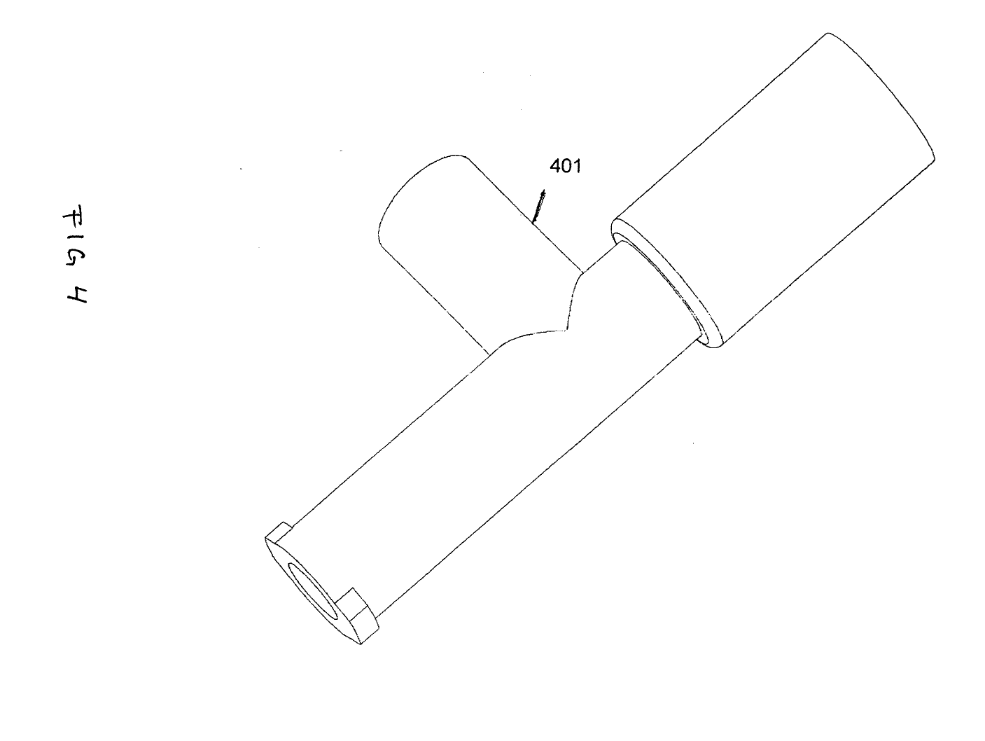

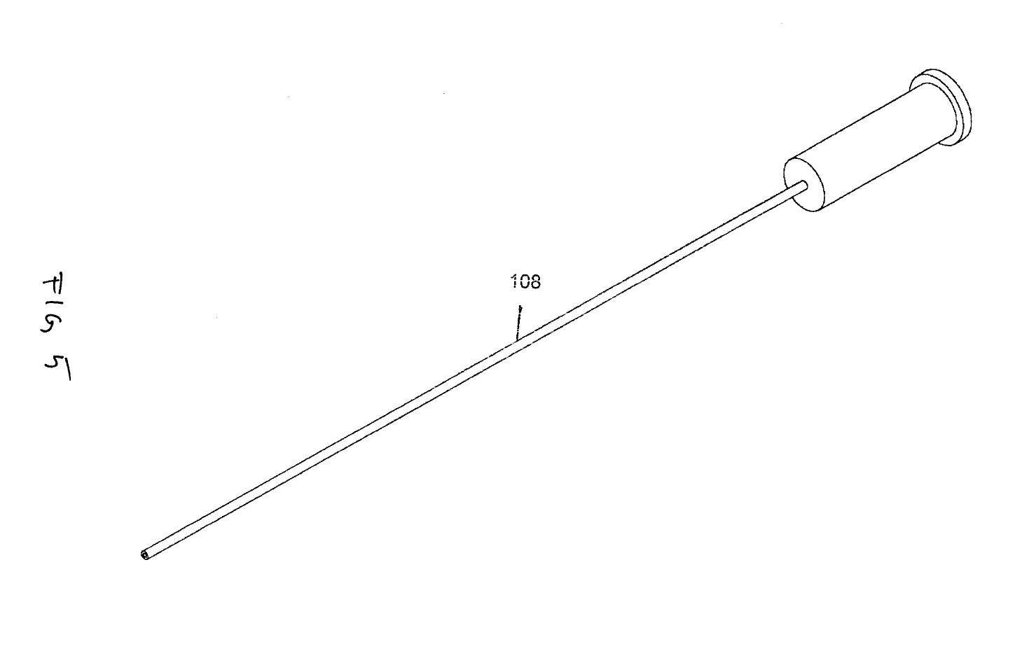

FIG. 3 shows the filter 301 of the PMEU light device according to an exemplary embodiment of the present invention. FIG. 4 shows the filter connector 401 according to an exemplary embodiment of the present invention. FIG. 5 shows the syringe 108 of the PMEU light device according to an exemplary embodiment of the present invention. The PMEU light device's gas (input and exhaust) filters 301 and the filter connectors 401 may be placed in the disposable bottom part. The gas filters 301 and the connector 401 specifically designed for them, are disposable so they may be firmly placed in the bottom part 107 so that the syringes 108 can be effortlessly installed in the connectors 401.

According to an exemplary embodiment the light version of the PMEU device (PMEU light) is suitable for cultivation and diagnosis of different microbes. Examples of such microbes are enterococci (E. faecalis, cgf group, E. hirae, E. cecorum, E. asini), E. coli and other coliforms, Vibrio cholerae, Yersinis sp. and other Enterobacteriaceae, klebsiella, Klebsiella pneumophila, campylobacter, C. jejuni, C. coli, Bacillus sp., Bacillus cereus, Streptococcus sp, Staphylococcus aureus and other staphylococci, Clostridium difficile and other clostridia, Mycobacterium sp, M. tuberculosis, salmonella and/or lactic acid bacteria, however not limited to these. Instead, according to an exemplary embodiment, the PMEU light device can be suitable for the cultivation and observation of a number of other beneficial or harmful microbes.

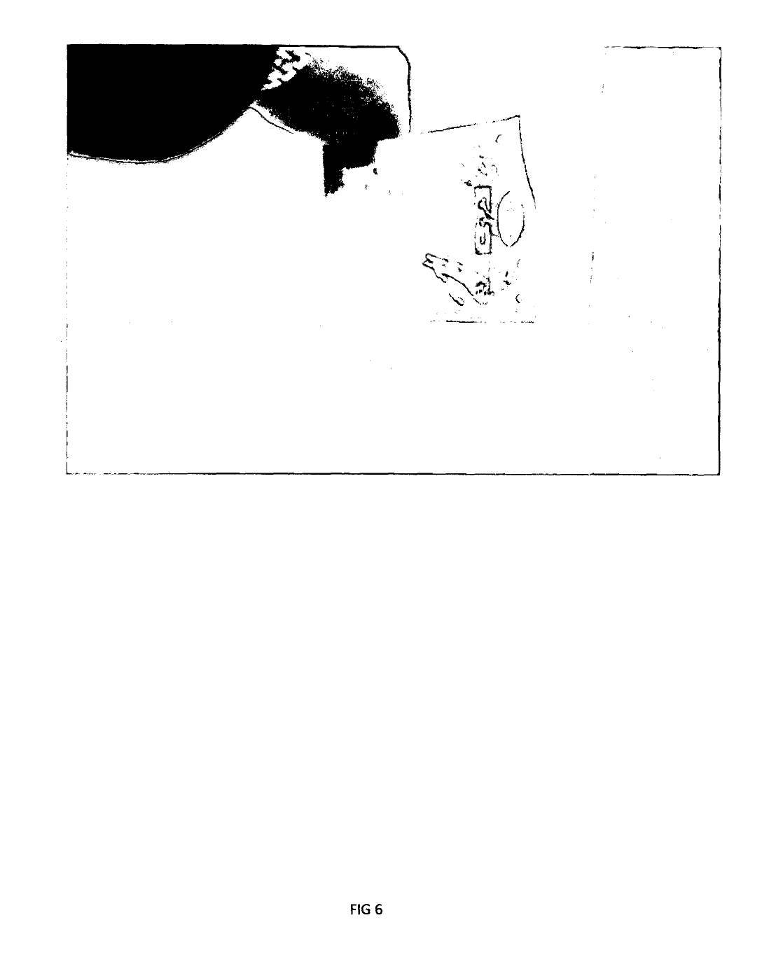

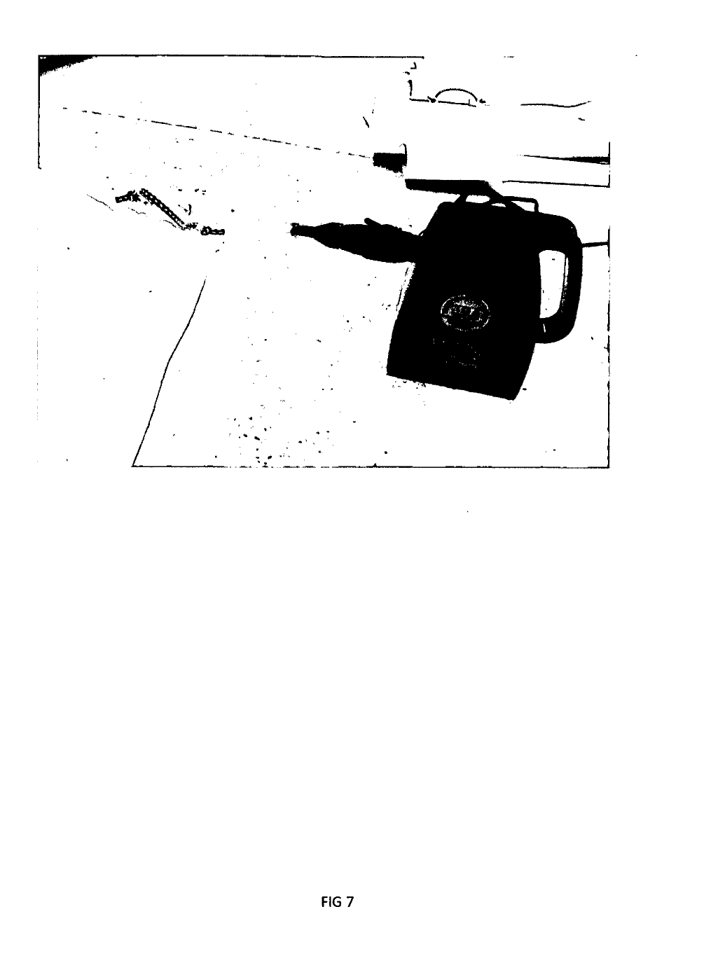

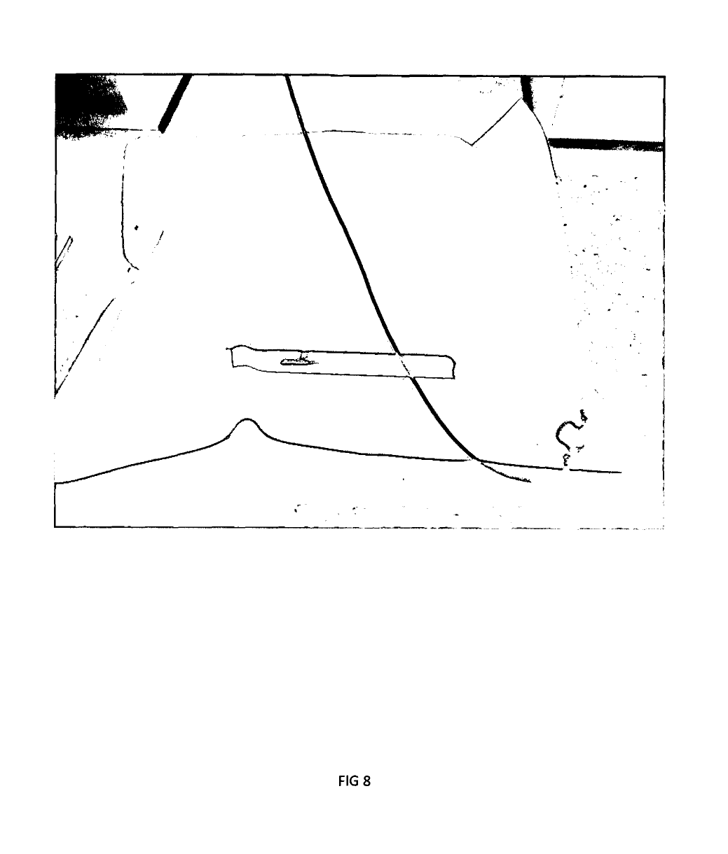

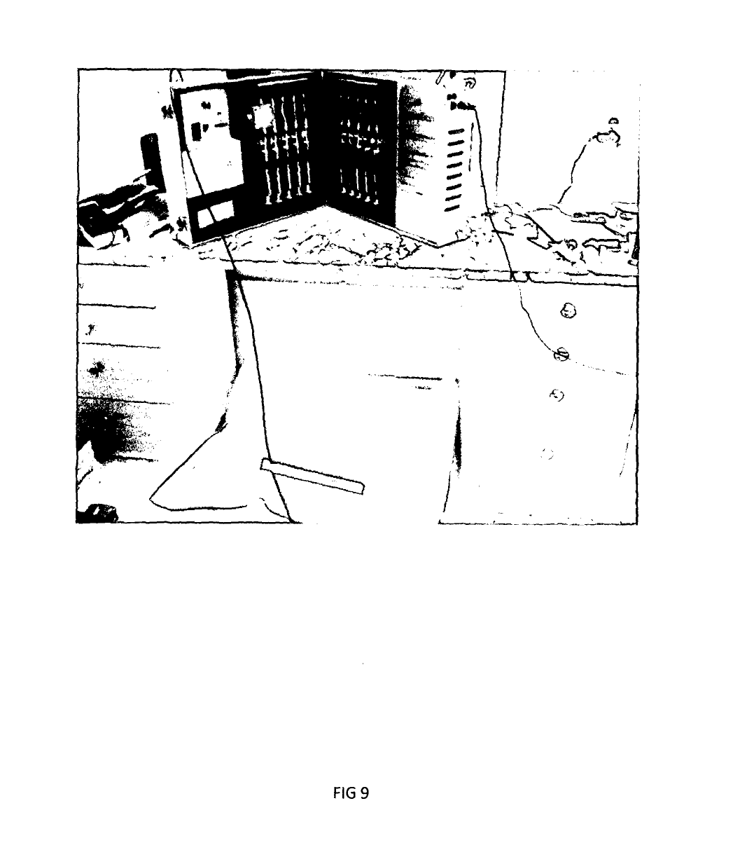

Thus offered is a microbial cultivation device that comprises the bottom part of the microbial cultivation device, each bottom part comprises a syringe holder, which is configured to receiving one or several syringes; the top part of the microbial cultivation device, each top part comprises:
one or more LED sensors and/or similar optical sensors for diagnosis of a sample in corresponding one or more sampling syringes.
thermoregulation unit for controlling the ambient temperature in the sampling syringes.
aeration unit for aerating the sampling syringes, and
I/O unit to provide a computer connection between the microbial cultivation device and a computer; wherein the top part is connected to the bottom part so that the top part is removable from the bottom part allowing for the disinfection of the top part and the disposal of the bottom part. It is also offered a microbial cultivation device, where the PMEU device or corresponding microbiological equipment is connected with a gas supply system as illustrated in FIGS. 6-9.

Additionally a microbial cultivation device is offered, wherein the top part of the microbial cultivation device can be disinfected and thus reused; and the bottom part of the microbial cultivation device is disposable and therefore has to be discarded.

Furthermore offered is a microbial cultivation device, wherein the top part of the microbial cultivation device comprises tools for controlling the aeration unit, thermoregulation unit, and/or sensors.

Furthermore offered is a microbial cultivation device, wherein the device is relevantly cylindrical in shape.

Furthermore offered is a microbial cultivation device, wherein the top part of the microbial cultivation device is at least partially in a protective encasing so that the top part of the device can be disinfected with disinfectant, hot steam, and/or UV-light.

Furthermore offered is a microbial cultivation device that is portable.

Furthermore offered is a microbial cultivation device that comprises a light version of a portable microbe enrichment unit PMEU device.

Furthermore offered is a microbial cultivation device that comprises a sensor bracket for receiving LED sensors and/or other similar optical sensors, each sensor bracket is configured for analysing samples in different sized sampling syringes.

Furthermore offered is a microbial cultivation device that is configured to sending an e-mail or text message alert if predetermined microbes are found in a sample.

Furthermore a bottom part of a microbial cultivation device is offered for the microbial cultivation device.

Furthermore a top part of a microbial cultivation device is offered for the microbial cultivation device.

Furthermore a disinfection device comprising tools for receiving the top part of the microbial cultivation device; tools for directing disinfectant and/or hot steam on the received top part of the microbial cultivation device for the disinfection of the microbial cultivation device is offered.

Furthermore a disinfection device comprising of a disinfection bath is offered.

Furthermore offered is a disinfection device comprising a heating element for producing hot vapour for the disinfection of the top part of the microbial cultivation device.

Furthermore offered is a microbiological cultivation and enrichment device and protocol with additional parts and steps for producing a specific gas atmosphere with lowered oxygen content, if required for the cultivation of microaerobic or anaerobic bacteria, or for achieving optimal conditions for any microbes. To professionals it is evident that as technology advances, the basic concept of the invention can be implemented in numerous different ways. Therefore the invention and its embodiments are not restricted to the examples illustrated above but can vary within the frame of the patent claim.

1-16. (canceled)

17: A microbial cultivation device comprising:
a bottom part including a container holder having a configuration capable of holding at least one sampling container selected from the group consisting of a syringe, and a culture vessel; and
a top part comprising:
at least one optical sensor capable of diagnosing at least one sample in said sampling container;
a thermoregulation unit having a configuration capable of controlling an ambient temperature in said sampling container;
an aeration unit having a configuration capable of aerating said sampling container; and
an I/O-unit having a configuration capable of enabling a computer connection between said microbial cultivation device and a computer;
wherein said top part is connectable to said bottom part so that said top part is removable from said bottom part.

18: The microbial cultivation device according to claim 17, wherein said top part is capable of being disinfected and reused, and said bottom part is capable of being discarded after use.

19: The microbial cultivation device according to claim 17, wherein said top part further comprising control tools having a configuration capable of controlling at least one of said aeration unit, said thermoregulation unit, and said optical sensor.

20: The microbial cultivation device according to claim 19, wherein said microbial cultivation device is substantially cylindrical in form.

21: The microbial cultivation device according to claim 20, wherein said top part is at least partially protected by an encasing so that said top part is disinfected with at least one selected from the group consisting of a disinfection gas, steam, and UV-light.

22: The microbial cultivation device according to claim 17, wherein said microbial cultivation device is a portable microbe enrichment unit (PMEU) having a light device.

23: The microbial cultivation device according to claim 17, wherein said top part further comprising a sensor bracket having a configuration capable of supporting said optical sensor, said sensor bracket has a configuration capable of positioning said optical sensor to analyze the sample from said sampling container.

24: The microbial cultivation device according to claim 23, wherein said container holder has a configuration capable of holding a plurality of said sampling container in a radial configuration, and wherein said sensor bracket includes a plurality of support arms radially extending out a longitudinal axis of said top part, with at least one of said optical sensor located at a distal end of each of said members, said sensor bracket and said container holder being configured that said optical sensor is positioned adjacent said sampling container, respectively, when said top part and said bottom part are connected.

25: The microbial cultivation device according to claim 17, wherein said computer has a configuration capable of sending an e-mail or text message alert if predetermined microbes are detected in the sample by said optical sensor.

26: The microbial cultivation device according to claim 17, wherein said bottom part further comprising at least one gas filter and at least one filter connector, said filter connector has a configuration capable of connecting with said gas filter and to receive a portion of said sampling container.

27: The microbial cultivation device according to claim 17 further comprising a disinfection device comprising:
at least one first tool having a configuration capable of receiving at least a portion of said top part;
at least one second tool having a configuration capable of directing at least one selected from the group consisting of a disinfecting gas, and steam on said top part to disinfect said top part;
at least one third tool having a configuration capable of directing UV-light on said top part to disinfect said top part.

28: The microbial cultivation device according to claim 27 further comprising a disinfection bath.

29: The microbial cultivation device according to claim 27 further comprising a heating element (203) for heating said disinfection device or to produce steam to disinfect said top part.

30: The microbial cultivation device according to claim 27 further comprising a gas producing equipment comprising:
at least one fourth tool having a configuration capable of filling a sack with a gas mixture; and
a means for leading said gas mixture into said sampling container;
wherein said sack is located outside said microbial cultivation device.
